(19) United States
(12) Patent Application Publication
(10) Pub. No.: US 2022/0047838 A1
(43) Pub. Date: Feb. 17, 2022

(54) One-Way CPAP/BiPAP Mask

(71) Applicant: IMRAN MINHAS, WEST CHESTER, OH (US)

(72) Inventor: IMRAN MINHAS, WEST CHESTER, OH (US)

(21) Appl. No.: 16/991,228

(22) Filed: Aug. 12, 2020

(51) Int. Cl. A61M 16/20; A61M 16/06; A61M 16/10; A61M 16/00

(52) U.S. Cl. CPC A61M 16/208; A61M 16/0666; A61M 2016/0027; A61M 16/0003; A61M 16/0644; A61M 16/1065

(57) ABSTRACT

An article of manufacture for a one-way CPAP/BiPAP mask is disclosed. The article includes a mask covering a mouth and nose of a user, an inlet for connecting to a CPAP/BiPAP machine, an exhalation port having an interlocking ring, and a removable filtering tube having an attachment end and an exhalation end. The removable filtering tube has an attachment port located at the attachment end and configured to connect to the interlocking ring of the exhalation port, a filtered exhalation opening located at the exhalation end, and a filter element located between the attachment port and the filtered exhalation opening, the filter permits air from within the mask leave the filtered exhalation opening while removing any pathogens present in the user's exhalations.

### TECHNICAL FIELD

This application relates in general to an article of manufacture for a personal protection device, and more specifically, to an article of manufacture for a one-way CPAP/BiPAP mask.

### BACKGROUND

In the beginning of the most recent COVID-19 virus crisis, a concern arose regarding the lack of available ventilators to treat patients. One contributing reason for the lack of ventilators was that patients who would otherwise benefit from a higher flow of oxygen via a CPAP or BiPAP machine could not safely use those devices because the virus present in their exhalation might contaminate the machine, room, and healthcare providers. After 5 L of nasal cannula oxygen patients typically were directed straight to a ventilator for continued treatment.

Furthermore, COVID-19 patients who are put on a ventilator often experience pulmonary barotrauma. This possible complication may benefit from a less aggressive oxygenation which will decrease the barotrauma incidence, and at the same, time allow for more oxygenation than a simple 5 L nasal cannula provides. Such a treatment regimen may hopefully put less stress on a patient's lungs, increase oxygenation, and theoretically provide better outcomes for the patient.

Therefore, a need exists for a new mask that permits patients to receive high flow oxygen via more readily available CPAP and BiPAP machines. With an improved mask concept, contaminated, virus-laden exhalation will be channeled through a filter before being released safely into a patient's room. This intervention may allow a larger pool of patients that otherwise would've gone straight to a ventilator to be allowed to utilize CPAP or BiPAP machine treatment with less barotrauma than a ventilator provides. The present invention attempts to address the shortcomings in the prior art systems according to the principles disclosed herein.

### SUMMARY

In accordance with the present invention, the above and other problems are solved by providing an article of manufacture for a one-way CPAP/BiPAP mask according to the principles and example embodiments disclosed herein.

In one embodiment, the present invention is an article of manufacture for a one-way CPAP/BiPAP mask. The article includes a mask covering a mouth and nose of a user, an inlet for connecting to a CPAP/BiPAP machine, an exhalation port having an interlocking ring, and a removable filtering tube having an attachment end and an exhalation end. The removable filtering tube has an attachment port located at the attachment end and configured to connect to the interlocking ring of the exhalation port, a filtered exhalation opening located at the exhalation end, and a filter element located between the attachment port and the filtered exhalation opening, the filter permits air from within the mask leave the filtered exhalation opening while removing any pathogens present in the user's exhalations.

In another aspect of the present invention, the article further includes an air valve coupled to the exhalation port and a pressure sensor coupled to the air valve and configured to open the air valve in a gradient fashion allowing exhalation from the user to exit the mask.

In another aspect of the present invention, the air valve comprises one of the following types of valves: butterfly, piston, and ball valves.

In another aspect of the present invention, the article also includes an actuator coupled to the pressure sensor and configured to adjust an amount of airflow able to escape the exhalation port.

In another aspect of the present invention, the mask operates as a CPAP/BiPAP mask when the actuator causes the air valve to remain open.

In another aspect of the present invention, the actuator having a set of different pressure points controlled by the pressure sensor, the set of different pressure points corresponding to pressure levels present in the mask at various points on the user's breathing patterns.

In another aspect of the present invention, the article further includes an adjustable head strap for holding the mask in place on the user.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only, and is not intended as a definition of the limits of the present invention.

### BRIEF DESCRIPTION OF THE DRAWINGS

Referring now to the drawings in which like reference numbers represent corresponding parts throughout:

FIG. 1 illustrates an example embodiment for an article of manufacture for a one-way CPAP/BiPAP mask according to the present invention.

FIG. 2 illustrates another example embodiment of an article of manufacture for a one-way CPAP/BiPAP mask according to the present invention.

FIG. 3 illustrates yet another example embodiment of an article of manufacture for a one-way CPAP/BiPAP mask according to the present invention.

### DETAILED DESCRIPTION

This application relates in general to an article of manufacture for a personal protection device, and more specifically, to an article of manufacture for a one-way CPAP/BiPAP mask according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. Thus, for example, reference to “a needle” includes reference to one or more of such needles and “etching” includes one or more of such steps. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps or components, but do not preclude the presence or addition of one or more other features, steps or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

As used herein, the term “about” means that dimensions, sizes, formulations, parameters, shapes, and other quantities and characteristics are not and need not be exact, but may be approximated and/or larger or smaller, as desired, reflecting tolerances, conversion factors, rounding off, measurement error and the like, and other factors known to those of skill. Further, unless otherwise stated, the term “about” shall expressly include “exactly.”

The terms “patient,” and “user” refer to an entity, e.g. a human, using the one-way CPAP/BiPAP mask including any filter elements associated with the invention. The term user herein refers to one or more users.

The term “invention” or “present invention” refers to the invention being applied for via the patent application with the title “One-Way CPAP/BiPAP Mask.” Invention may be used interchangeably with filter mask.

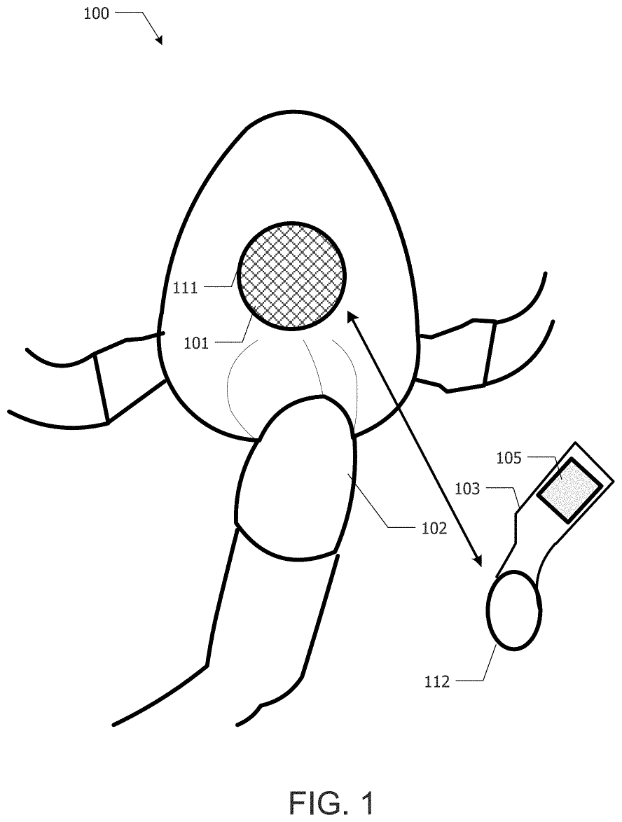

In general, the present disclosure relates to an article of manufacture for a personal protection device. To better understand the present invention, FIG. 1 illustrates an example embodiment for an article of manufacture for a one-way CPAP/BiPAP mask 100 according to the present invention. As the current COVID-19 virus pandemic evolves, it has become apparent that patients require appropriate oxygen ventilation. Initially, a recommendation was followed that any treatment beyond 5 L nasal cannula oxygenation should indicate intubation. Any steps in-between including high flow oxygen, CPAP treatment, and BiPAP treatment were skipped because the mask design spewed virus-laden exhalation throughout the patient area, endangering patients, healthcare providers, and staff. Due to this, medical centers were faced with acute shortages of ventilators for intubation. Recommendations now have changes where ventilators only are recommended for ARDS (Acute Respiratory Distress Syndrome) patients.

Most CPAP/BiPAP masks are more or less the same design: the mask 100 has holes of an exhalation port 101 to allow for excess pressure to escape into the atmosphere along with, unfortunately, any pathogens such as COVID-19. The present invention, by contrast, has a single entry point 102 with positive pressure from the CPAP and BiPAP machine. The mask 100 also has an interlocking attachment ring 111 about the outer edge of the exhalation port 101.

The interlocking attachment ring 111 may couple to an attachment port 112 of a filtering tube 103 in order for the filtering tube 103 to cover the exhalation port 101. A filter 105 may be installed within the filtering tube 103 causing any exhalation from the patient must pass through the filter 105 before exiting the mask 100 into the room environment.

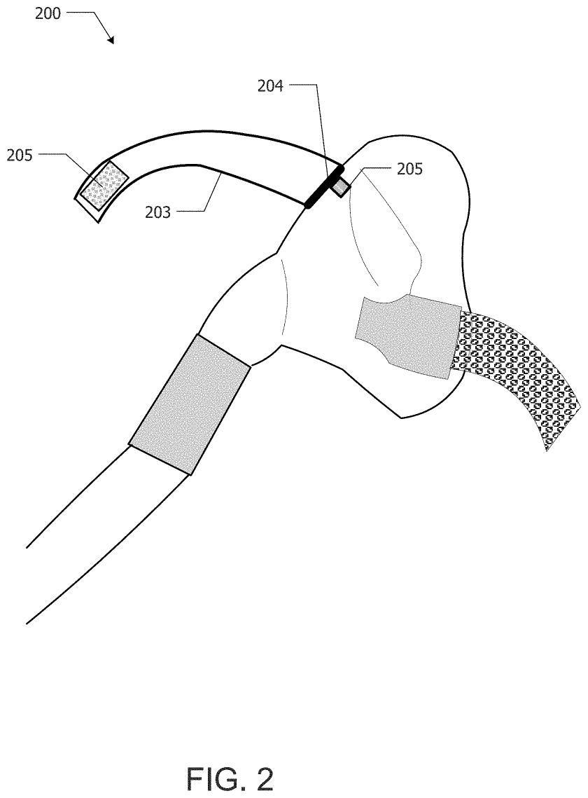

In current masks, an inlet 102 is present where the pressure generation from the CPAP device enters the mask 100. A perforated area 101 on the mask 100 above this inlet 101 allows for the exhaust to exit. The present invention differs from prior solutions, such as other vent-less mask designs, because the present invention's emphasis is on the exhaust vent side of the mask, not the pressure generator side. The present invention replaces the perforated area and attaches a tube with a valve as shown in FIG. 2.

FIG. 2 illustrates another example embodiment of an article of manufacture for a one-way CPAP/BiPAP mask according to the present invention. The present invention comprises a mask 200 that utilizes a filtering tube 203. In that tube 203, a pressure sensor 205 linked to a butterfly valve 204 that will open in a gradient fashion or fully to allow the exhalation from the patient to be channeled through a filter. The valve 204 may consist of any mechanism that can be adjusted, including butterfly, piston, and ball valves.

By using the valve 204, patients may be given breathing treatments without worry that the virus being blown into the atmosphere hence contaminating the room, the equipment, or the health care personnel. Having the valve 204 open and shut via pressure sensor 205 allows a more effective inhalation medication. As the patient inhales, the pressure sensor 205 allows the valve 204 to shut and the full dose of breathing treatment goes into the lungs. As a patient exhales the sensor 205 picks up on the pressure change and opens for expulsion of breath. Otherwise what happens is this medication is being fed into the mask continuously whether the patient is inhaling or not it will attempt to escape outside the vent holes on the mask.

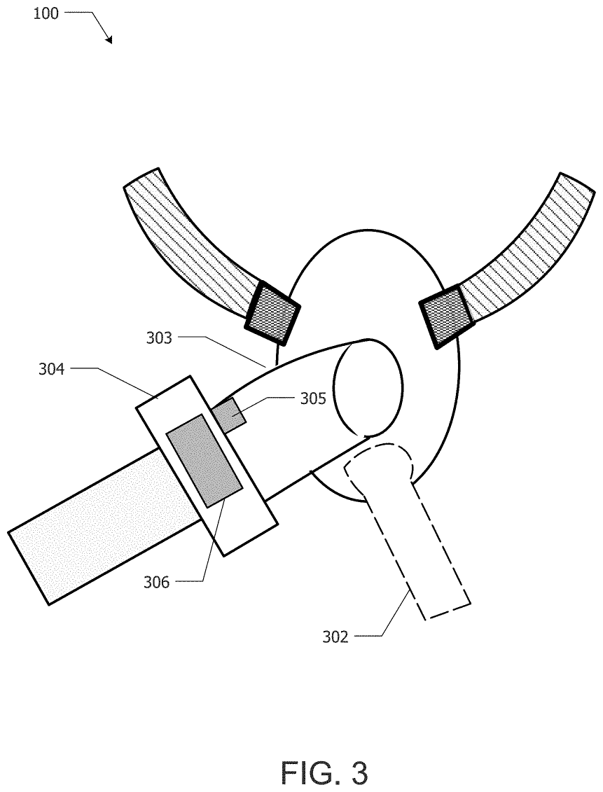

FIG. 3 illustrates yet another example embodiment of an article of manufacture for a one-way CPAP/BiPAP mask according to the present invention. The present invention comprises a mask 300 that includes an intake hose 302, an exhalation hose 303, and a valve 304 which may include a pressure sensor 305 with an actuator 306 that can adjust the valve 304. The pressure sensor 305 allows for flexibility in defining a treatment regimen. If the valve 304 is held in an open position it will operate as a normal CPAP mask. With variable adjustability of the valve 304, the mask may be set to different pressure points that may correspond with the patient's individual breathing patterns as opposed to just a preset machine. This process may also produce a similar effect as a BiPAP machine. The key is a pressure sensor 305 in the outlet which can modulate the opening degree of the valve 304.

Once this one-way CPAP/BiPAP mask 300 is operational, it will ease the pressure for ventilators and allow physicians more flexibility prescribe more appropriate and different modalities that will reduce the incidence of pulmonary barotrauma associated with ventilators. Use of the CPAP/BiPAP mask 300 in a time of crisis, a shortage of equipment may be lessened to the extend the functionality of the CPAP device functioning as a BiPAP device based on programming the valve 305 to open and close on certain pressures. So typically, on a BiPAP device, the machine sets a rhythmic variation in pressure which the patient has to match the machine to breathe properly. When there is a shortage of all kinds of equipment, the CPAP machine may be converted into a BiPAP machine. Because of the pressure sensor 305, the valve may be set to open until a certain pressure is attained. This pressure is lower than a typical CPAP device setting which allows for the exhalation and therefore, improve ventilation of the patient. This functionality allows for more extended time for the patient to receive treatment while utilizing a gentler ventilating system via a CPAP or BiPAP vs intubation and ventilator. Theoretically at least, this functionality may allow for higher pressures to be given to support lung ventilation than a CPAP device and BiPAP device otherwise provides without being as harsh as a ventilator.

Even though particular combinations of features are recited in the present application, these combinations are not intended to limit the disclosure of the invention. In fact, many of these features may be combined in ways not specifically recited in this application. In other words, any of the features mentioned in this application may be included in this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed:

1. An article of manufacture for providing a one-way CPAP/BiPAP mask, the article comprises:
a mask covering a mouth and nose of a user;
an inlet for connecting to a CPAP/BiPAP machine;
an exhalation port having an interlocking ring; and
a removable filtering tube having an attachment end and an exhalation end comprising:
an attachment port located at the attachment end and configured to connect to the interlocking ring of the exhalation port;
a filtered exhalation opening located at the exhalation end; and
a filter element located between the attachment port and the filtered exhalation opening, the filter permits air from within the mask leave the filtered exhalation opening while removing any pathogens present in the user's exhalations.

2. The article of manufacture according to claim 1, wherein the article further comprises:
an air valve coupled to the exhalation port; and
a pressure sensor coupled to the air valve and configured to open the air valve in a gradient fashion allowing exhalation from the user to exit the mask.

3. The article of manufacture according to claim 2, wherein the air valve comprises one of the following types of valves: butterfly, piston, and ball valves.

4. The article of manufacture according to claim 1, wherein the article further comprises an actuator coupled to the pressure sensor and configured to adjust an amount of airflow able to escape the exhalation port.

5. The article of manufacture according to claim 4, wherein the mask operates as a CPAP/BiPAP mask when the actuator causes the air valve to remain open.

6. The article of manufacture according to claim 1, wherein the actuator having a set of different pressure points controlled by the pressure sensor, the set of different pressure points corresponding to pressure levels present in the mask at various points on the user's breathing patterns.

7. The article of manufacture according to claim 1, wherein the article further comprises an adjustable head strap for holding the mask in place on the user.
